(19) United States
(12) Patent Application Publication
(10) Pub. No.: US 2005/0043626 A1
(43) Pub. Date: Feb. 24, 2005

(54) Apparatus and method for an ultrasonic medical device engaging a flexible material

(75) Inventors: Rebecca I. Marciante, North Reading, MA (US); Bradley A Hare, Chelmsford, MA (US); Robert A. Rabiner, North Reading, MA (US); Anthony W. O'Leary, Walpole, MA (US); Mark J. Varady, Holliston, MA (US); Janniah S. Prasad, Norwalk, CT (US); Anita J. Chuang, Cambridge, MA (US)

Correspondence Address: PALMER & DODGE, LLP;RICHARD B. SMITH, 111 HUNTINGTON AVENUE, BOSTON, MA, 02199, US

(73) Assignee: OmniSonics Medical Technologies, Inc.

(21) Appl. No.: 10/646,408

(22) Filed: Aug. 22, 2003

(51) Int. Cl. A61B 8/14

(52) U.S. Cl. 600/459

(57) ABSTRACT

The present invention provides an apparatus and a method for an ultrasonic medical device having a flexible material engaging an ultrasonic probe. The flexible material surrounds a portion of a longitudinal axis of an ultrasonic probe of the ultrasonic medical device. The flexible material may extend beyond a probe tip. The flexible material cushions a tip of the ultrasonic probe and reduces the stresses on the ultrasonic probe as the ultrasonic probe is navigated within the vasculature. The ultrasonic probe may be shaped to increase a radial span of the ultrasonic medical device. In a preferred embodiment of the present invention, the flexible material comprises a polymer material. Additionally, the flexible material may have a high radiopacity.

### RELATED APPLICATIONS

None.

### FIELD OF THE INVENTION

The present invention relates to an ultrasonic medical device, and more particularly to an apparatus and method for an ultrasonic medical device engaging a flexible material used to remove a biological material.

### BACKGROUND OF THE INVENTION

Vascular occlusive disease affects millions of individuals worldwide and is characterized by a dangerous blockage of blood vessels. Vascular occlusive disease includes thrombosed hemodialysis grafts, peripheral artery disease, deep vein thrombosis, coronary artery disease, heart attack and stroke. Vascular occlusions (including, but not limited to, clots, intravascular blood clots or thrombus, occlusional deposits, such as calcium deposits, fatty deposits, atherosclerotic plaque, cholesterol buildup, fibrous material buildup and arterial stenoses) result in the restriction or blockage of blood flow in the vessels in which they occur. Occlusions result in oxygen deprivation (“ischemia”) of tissues supplied by these blood vessels. Prolonged ischemia results in permanent damage of tissues which can lead to limb loss, myocardial infarction, stroke, or death. Targets for occlusion include coronary arteries, peripheral arteries and other blood vessels.

The disruption of an occlusion can be affected by pharmacological agents, mechanical methods, ultrasonic methods or combinations of all three. Many procedures involve inserting a medical device into a vasculature of the body. Medical devices include, but are not limited to, probes, catheters, wires, tubes and similar devices. In some cases, the medical device delivers a pharmacological agent to the site of the occlusion.

Navigation of a probe within a vasculature of a body to a site of an occlusion can be a challenging process for a surgeon. The difficulty of the navigation lies in the path of the particular vasculature that is being navigated, the degree of blockage of the occlusion of biological material and the physical properties of the probe. Probes need to have a degree of rigidity in order for a surgeon to be able to control the insertion process through the tortuous paths of the vasculature. Often times, a torque is applied to the probe to move the probe through the vasculature. In addition, probes need to have a degree of flexibility so the probe can flex, bend and curve according to the path of the vasculature. The flexibility also reduces the potential risk of damage to the healthy tissue as the probe is being navigated within the vasculature.

Navigation of the probe through the vasculatures of the body is difficult due to the high stresses that are required to bend the probe as the user applies force and/or torque to move the probe to the treatment site. As the diameter of the probe increases, it is more difficult to bend the probe. Applications where the probe is used in a vasculature deep within the body present the largest challenge for the user. The high stresses that are imparted to the walls of the vasculature in the body as the probe is moved to the treatment site can weaken the vasculature. Often times, the probe is moved to the treatment site after a series of probe withdrawals and probe re-insertions, with each withdrawal and insertion of the probe potentially weakening the vasculature. In order to alleviate these problems, it is desirable that the geometry of the distal end of the probe be flexible enough to traverse within the anatomy of the vasculature. In addition, there is a need in the art for a probe that increases a surface area of the probe in communication with an occlusion.

In addition to the weakening of the vasculature and the need to reduce stresses, ultrasonic probes with a large diameter require a higher amount of power in order to cause vibration in the probe. It is desirable to minimize the power during the ultrasonic vibration of the probe, since increased power levels lead to excess heating of the probe, potential damage to the vasculature and the patient, and functional limitations of the probe. Straight probes used in the ablation of an occluded material also require a high power output to maximize the effect of the ultrasonic energy and require long treatment times for the ablation of the occluded material. Therefore, there is a need in the art for an ultrasonic probe that increases the surface area in communication with the occlusion so the required power to ablate the occlusion can be minimized to eliminate potential damage to the vasculature and the patient.

U.S. Pat. No. 5,235,964 to Abenaim discloses a reusable probe apparatus with a double sleeve probe housing. The Abenaim device is used for housing a transesophageal probe or comparable medical instrumentation and is used for insertion to the stomach via the mouth and throat for subsequent manipulation. The Abenaim device is limited to specific vasculatures in the body and is difficult to maneuver through the vasculature. Therefore, there is a need in the art for an apparatus and a method of delivering an ultrasonic probe to a site of an occlusion within a vasculature of a body that is not limited to specific vasculatures, can be shaped to be navigated within the vasculature, does not damage the vasculature and can be used to ablate an occlusion in the vasculature.

U.S. Pat. No. 5,402,799 to Colon et al. discloses a guidewire having a coil at a distal end. The Colon et al. device comprises a unitary core wire comprising nickel and titanium alloy, a distal portion with a ribbon tip comprising nickel and titanium alloy and a metallic coil that is positioned along the outside surface of the distal tip portion. The irregular surface from the metallic coils of the Colon et al. device can cause damage to the vessel as the device is navigated through the vasculature. The Colon et al. metallic coils provide a rough surface that can perforate or damage the vessel. Therefore, there is a need in the art for an apparatus and a method of delivering an ultrasonic probe to a site of an occlusion within a vasculature that does not damage the vasculature, can be easily navigated within the vasculature in a time efficient manner and can be used to ablate occlusions in the vasculature.

U.S. Pat. No. 4,748,986 to Morrison et al. discloses a guide wire comprising a metallic element with a coil concentrically secured to the metallic element. The element and coils are composed of metallic materials such as stainless steel. The metallic coils of the Morrison et al. device provide an irregular and rough surface that can damage the vessel as the device is navigated through the vessel. The outer diameter of the distal end of the Morrison et al. device limits the use of the device to specific large vasculatures of the body. Therefore, there is a need in the art for an apparatus and a method of delivering an ultrasonic probe to a site of an occlusion within a vasculature of a body that does not damage the vasculature, is not limited to specific vasculatures, can be easily navigated within the vasculature in a time efficient manner and can be used to ablate occlusions in the vasculature.

The prior art does not solve the problem of providing an ultrasonic medical device that can be navigated within a vasculature in a simple, safe and time efficient manner. The prior art devices are limited in the vasculatures the prior art devices can be used in, and the prior art devices inflict high stresses on the vasculature and the device itself. Prior art devices lack the flexibility to be safely moved within the vasculature and are limited in how the prior art devices can be shaped. The prior art devices require a high amount of power that can damage the vasculatures and require long treatment times that can adversely affect healthy tissue in the patient. Therefore, there remains a need in the art for an apparatus and method of delivering an ultrasonic probe to a site of an occlusion within a vasculature of a body that does not damage the vasculature, is not limited to specific vasculatures, can be easily navigated within the vasculature in a time efficient manner and can be used to ablate occlusions in the vasculature.

### SUMMARY OF THE INVENTION

The present invention is an ultrasonic medical device engaging a flexible material used to ablate a biological material. The ultrasonic medical device includes an ultrasonic probe having a proximal end, a distal end and a longitudinal axis therebetween. A flexible material surrounds at least a portion of the longitudinal axis of the ultrasonic probe. The flexible material may extend beyond a probe tip. The portion of the longitudinal axis of the ultrasonic probe with the flexible material may be shaped to increase a radial span of the ultrasonic medical device. The flexible material protects a vasculature as the ultrasonic probe is moved through the vasculature. The flexible material may comprise a material of high radiopacity to enhance the visibility of the ultrasonic medical device during certain medical procedures. The flexible material may engage various locations along the longitudinal axis of the ultrasonic probe.

The present invention is an ultrasonic medical device for removing a biological material. The ultrasonic medical device includes an elongated ultrasonic probe having a proximal end, a distal end and a longitudinal axis therebetween. In one embodiment, a connecting segment engages the distal end of the elongated ultrasonic probe and a flexible material extends from the connecting segment. The flexible material is more flexible than the elongated ultrasonic probe. The flexible material cushions a tip of the ultrasonic probe as the ultrasonic probe is moved through a vasculature.

The present invention provides a method of moving an ultrasonic probe along a path in a vasculature of a body to the site of an occlusion. A flexible material is engaged to the ultrasonic probe and the ultrasonic probe with the flexible material is inserted into the vasculature. The ultrasonic probe is advanced in the vasculature until the flexible material contacts a wall of the vasculature to allow the ultrasonic probe to bend along the path in the vasculature. The ultrasonic probe is then moved further within the vasculature.

In a preferred embodiment of the present invention, a flexible material engages a tip of the ultrasonic probe. The flexible material extends beyond the probe tip. In another embodiment, the flexible material ends at the probe tip. In another embodiment, the flexible material may be located anywhere along the longitudinal axis of the ultrasonic probe. The ultrasonic probe with the flexible material is inserted into a vasculature and advanced within the vasculature. An ultrasonic energy source is activated to provide an ultrasonic energy to the ultrasonic probe to ablate the biological material. The ultrasonic probe with the flexible material may be shaped to increase a radial span of the ultrasonic probe or to allow for steering within the vessel.

The present invention provides a method for adhering a flexible material to an ultrasonic medical device comprising: providing the flexible material to be adhered to the ultrasonic medical device; engaging the flexible material to the ultrasonic medical device; heating the flexible material engaged to the ultrasonic medical device with a heat source causing the flexible material to melt; and cooling the flexible material engaged to the ultrasonic medical device. In an embodiment, the flexible material is a polymer. In an embodiment, the flexible material comprises a high radiopacity.

The present invention provides an apparatus and a method for an ultrasonic probe engaging a flexible material. The flexible material provides the flexibility to move the ultrasonic probe in the vasculature of the body to remove an occlusion while protecting the vasculature without adversely affecting the functionality of the ultrasonic probe. The flexible material may comprise a material of high radiopacity to enhance the visibility of the ultrasonic probe during certain medical procedures. The present invention provides an ultrasonic probe with a flexible material that is simple, user-friendly, reliable and cost effective.

### BRIEF DESCRIPTION OF THE DRAWINGS

The present invention will be further explained with reference to the attached drawings, wherein like structures are referred to by like numerals throughout the several views. The drawings shown are not necessarily to scale, with emphasis instead generally being placed upon illustrating the principles of the present invention.

FIG. 1 shows a side plan view of a preferred embodiment of an ultrasonic medical device of the present invention capable of operating in a transverse mode with a tip of a longitudinal axis of an ultrasonic probe engaged by a flexible material extending beyond the tip of the ultrasonic probe.

FIG. 2 shows a fragmentary side plan view of a preferred embodiment of an ultrasonic probe of the present invention with a flexible material extending from a tip of the ultrasonic probe.

FIG. 3 shows a cross section view of a connecting segment of an ultrasonic probe and a flexible material taken along line A-A of FIG. 2.

FIG. 4 shows a cross section view of a flexible material extending from an ultrasonic probe taken along line B-B of FIG. 2.

FIG. 5 shows a side plan view of an alternative embodiment of the ultrasonic medical device of the present invention with a portion of a longitudinal axis of an ultrasonic probe surrounded by a flexible material.

FIG. 6 shows a fragmentary side plan view of an alternative embodiment of the present invention with a portion of a longitudinal axis of the ultrasonic probe surrounded by a flexible material.

FIG. 7 shows a cross section view of a portion of a longitudinal axis of the ultrasonic probe surrounded by a flexible material taken along line C-C of FIG. 6 and FIG. 9.

FIG. 8 shows a side plan view of an alternative embodiment of the present invention of an ultrasonic medical device capable of operating in a transverse mode with a longitudinal axis of an ultrasonic probe surrounded by a flexible material.

FIG. 9 shows a fragmentary side plan view of an alternative embodiment of an ultrasonic probe of the present invention with a longitudinal axis of the ultrasonic probe surrounded by a flexible material.

FIG. 10 shows a fragmentary side plan view of an ultrasonic probe of the present invention with a portion of a longitudinal axis of the ultrasonic probe surrounded by a flexible material having a curved shape and the flexible material extending beyond the tip of the ultrasonic probe.

FIG. 11 shows a fragmentary side plan view of an alternative embodiment of an ultrasonic probe of the present invention with a portion of a longitudinal axis of the ultrasonic probe surrounded by a flexible material bent at an angle to a longitudinal axis of the ultrasonic probe.

FIG. 12 shows a fragmentary side plan view of an alternative embodiment of an ultrasonic probe of the present invention located at a bend in a vasculature and proximal to an occlusion with a tip of the ultrasonic probe engaged by a flexible material extending beyond the tip of the ultrasonic probe.

FIG. 13 shows a fragmentary side plan view of an alternative embodiment of an ultrasonic probe of the present invention located at a bend in a vasculature showing a plurality of transverse nodes and a plurality of transverse anti-nodes along a portion of a longitudinal axis of the ultrasonic probe.

FIG. 14 shows a fragmentary side plan view of an alternative embodiment of an ultrasonic probe of the present invention with a flexible material engaging the ultrasonic probe by a process of heat shrinking.

FIG. 15 shows a cross section view of a connecting segment of an ultrasonic probe taken along line D-D of FIG. 14.

FIG. 16 shows a fragmentary side plan view of an alternative embodiment of an ultrasonic probe of the present invention with a flexible material located at a plurality of locations along a longitudinal axis of the ultrasonic probe.

While the above-identified drawings set forth preferred embodiments of the present invention, other embodiments of the present invention are also contemplated, as noted in the discussion. This disclosure presents illustrative embodiments of the present invention by way of representation and not limitation. Numerous other modifications and embodiments can be devised by those skilled in the art which fall within the scope and spirit of the principles of the present invention.

### DETAILED DESCRIPTION

The present invention provides an apparatus and a method for using an ultrasonic medical device comprising an ultrasonic probe with a flexible material surrounding at least a portion of a longitudinal axis of the ultrasonic probe to ablate an occlusion. In a preferred embodiment of the present invention, a flexible material surrounds a portion of the longitudinal axis of the ultrasonic probe. The flexible material comprises a material of high radiopacity. In addition, the flexible material may extend beyond a tip of the ultrasonic probe. The ultrasonic probe with the surrounding flexible material may be shaped to increase a radial span of the ultrasonic medical device. In another embodiment of the present invention, the flexible material engages a portion of the longitudinal axis of the ultrasonic probe. The flexible material of the ultrasonic probe allows the ultrasonic probe to bend along a path in a vasculature. The flexible material reduces the stresses on the ultrasonic probe and prevents harm to the vasculature as the flexible material contacts a wall of the vasculature as the ultrasonic probe is moved along the vasculature. In another embodiment of the present invention, the flexible material surrounds the ultrasonic probe from the proximal end to the distal end.

The following terms and definitions are used herein:

“Ablate” as used herein refers to removing, clearing, destroying or taking away a biological material. “Ablation” as used herein refers to a removal, clearance, destruction, or taking away of the biological material.

“Node” as used herein refers to a region of a minimum energy emitted by an ultrasonic probe at or proximal to a specific location along a longitudinal axis of the ultrasonic probe.

“Anti-node” as used herein refers to a region of a maximum energy emitted by an ultrasonic probe at or proximal to a specific location along a longitudinal axis of the ultrasonic probe.

“Probe” as used herein refers to a device capable of propagating an energy emitted by the ultrasonic energy source along a longitudinal axis of the probe, resolving the energy into an effective cavitational energy at a specific resonance (defined by a plurality of nodes and a plurality of anti-nodes along an “active area” of the probe) and is capable of an acoustic impedance transformation of electrical energy to a mechanical energy.

“Transverse” as used herein refers to a vibration of a probe not parallel to a longitudinal axis of the probe. A “transverse wave” as used herein is a wave propagated along the probe in which a direction of a disturbance at a plurality of points of a medium is not parallel to a wave vector.

“Biological material” as used herein refers to a collection of a matter including, but not limited to, a group of similar cells, intravascular blood clots or thrombus, fibrin, calcified plaque, calcium deposits, occlusional deposits, atherosclerotic plaque, fatty deposits, adipose tissues, atherosclerotic cholesterol buildup, fibrous material buildup, arterial stenoses, minerals, high water content tissues, platelets, cellular debris, wastes and other occlusive materials.

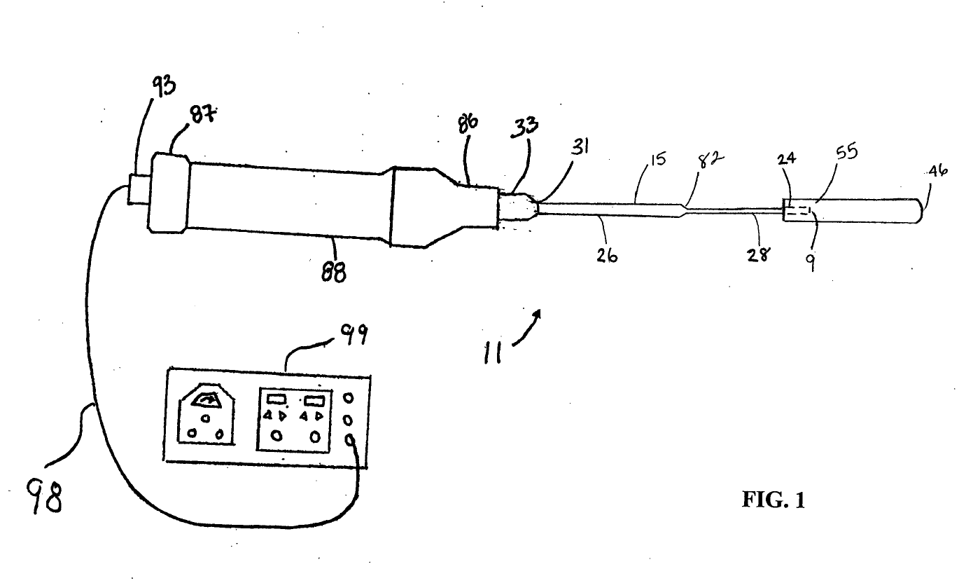

An ultrasonic medical device engaging a flexible material is shown generally at 11 in FIG. 1. The ultrasonic medical device 11 includes an ultrasonic probe 15 which is coupled to an ultrasonic energy source or generator 99 for the production of an ultrasonic energy. A handle 88, comprising a proximal end 87 and a distal end 86, surrounds a transducer (not visible) within the handle 88. The transducer, having a first end engaging the ultrasonic energy source 99 and a second end engaging a proximal end 31 of the ultrasonic probe 15, transmits the ultrasonic energy to the ultrasonic probe 15. A connector 93 and a connecting wire 98 engage the ultrasonic energy source 99 to the transducer. The ultrasonic probe 15 includes the proximal end 31, and a distal end 24 that ends in a probe tip 9. In a preferred embodiment of the present invention, a flexible material 55 surrounds the probe tip 9 of the ultrasonic probe 15 and extends beyond the probe tip 9.

A quick attachment-detachment system 33 that engages the proximal end 31 of the ultrasonic probe 15 to the transducer within the handle 88 is illustrated generally in FIG. 1. An ultrasonic probe device with a quick attachment-detachment system is described in the Assignee's co-pending patent applications U.S. Ser. No. 09/975,725; U.S. Ser. No. 10/268,487; and U.S. Ser. No. 10/268,843, and the entirety of all these applications are hereby incorporated herein by reference.

In a preferred embodiment of the present invention, the ultrasonic probe 15 is a wire. In another embodiment of the present invention, the ultrasonic probe 15 is elongated. In a preferred embodiment of the present invention, the diameter of the ultrasonic probe 15 decreases from the first defined interval 26 to the second defined interval 28 along the longitudinal axis of the ultrasonic probe 15 over an at least one diameter transition 82. In another embodiment of the present invention, the diameter of the ultrasonic probe 15 decreases at greater than two defined intervals. In a preferred embodiment of the present invention, the diameter transitions 82 of the ultrasonic probe 15 are tapered to gradually change the diameter from the proximal end 31 to the distal end 24 along the longitudinal axis of the ultrasonic probe 15. In another embodiment of the present invention, the diameter transitions 82 of the ultrasonic probe 15 are stepwise to change the diameter from the proximal end 31 to the distal end 24 along the longitudinal axis of the ultrasonic probe 15. Those skilled in the art will recognize that there can be any number of defined intervals and diameter transitions, and that the diameter transitions can be of any shape known in the art and be within the spirit and scope of the present invention.

In a preferred embodiment of the present invention, a cross section of the ultrasonic probe 15 is approximately circular. In other embodiments of the present invention, a shape of the cross section of the ultrasonic probe 15 includes, but is not limited to, square, trapezoidal, oval, triangular, circular with a flat spot and similar cross sections. Those skilled in the art will recognize that other cross sectional geometric configurations known in the art would be within the spirit and scope of the present invention.

The probe tip 9 can be any shape including, but not limited to, curved, a ball or larger shapes. In one embodiment of the present invention, the ultrasonic energy source 99 is a physical part of the ultrasonic medical device 11. In another embodiment of the present invention, the ultrasonic energy source 99 is not an integral part of the ultrasonic medical device 11.

The ultrasonic probe 15 is inserted into the vasculature and may be disposed of after use. In a preferred embodiment of the present invention, the ultrasonic probe 15 is for a single use and on a single patient. In a preferred embodiment of the present invention, the ultrasonic probe 15 is disposable. In another embodiment of the present invention, the ultrasonic probe 15 can be used multiple times.

In a preferred embodiment of the present invention, the ultrasonic probe 15 comprises titanium or a titanium alloy. Titanium is a strong, flexible, low density, low radiopacity and easily fabricated metal that is used as a structural material. Titanium and its alloys have excellent corrosion resistance in many environments and have good elevated temperature properties. In another embodiment of the present invention, the ultrasonic probe 15 comprises stainless steel. In another embodiment of the present invention, the ultrasonic probe 15 comprises a combination of titanium and stainless steel. Those skilled in the art will recognize that the ultrasonic probe can be comprised of many materials known in the art and be within the spirit and scope of the present invention.

In a preferred embodiment of the present invention, the ultrasonic probe 15 has a small diameter. In a preferred embodiment of the present invention, the diameter of the ultrasonic probe 15 gradually decreases from the proximal end 31 to the distal end 24. In an embodiment of the present invention, the diameter of the distal end 24 of the ultrasonic probe 15 is about 0.004 inches. In another embodiment of the present invention, the diameter of the distal end 24 of the ultrasonic probe 15 is about 0.015 inches. In other embodiments of the present invention, the diameter of the distal end 24 of the ultrasonic probe 15 varies between about 0.003 inches and about 0.025 inches. Those skilled in the art will recognize an ultrasonic probe 15 can have a diameter at the distal end 24 smaller than about 0.003 inches, larger than about 0.025 inches, and between about 0.003 inches and about 0.025 inches and be within the spirit and scope of the present invention.

In an embodiment of the present invention, the diameter of the proximal end 31 of the ultrasonic probe 15 is about 0.012 inches. In another embodiment of the present invention, the diameter of the proximal end 31 of the ultrasonic probe 15 is about 0.025 inches. In other embodiments of the present invention, the diameter of the proximal end 31 of the ultrasonic probe 15 varies between about 0.003 inches and about 0.025 inches. Those skilled in the art will recognize the ultrasonic probe 15 can have a diameter at the proximal end 31 smaller than about 0.003 inches, larger than about 0.025 inches, and between about 0.003 inches and about 0.025 inches and be within the spirit and scope of the present invention.

In an embodiment of the present invention, the diameter of the ultrasonic probe 15 is approximately uniform from the proximal end 31 to the distal end 24 of the ultrasonic probe 15. In another embodiment of the present invention, the diameter of the ultrasonic probe 15 gradually decreases from the proximal end 31 to the distal end 24. In an embodiment of the present invention, the ultrasonic probe 15 may resemble a wire. In an embodiment of the present invention, the gradual change of the diameter from the proximal end 31 to the distal end 24 occurs over the at least one diameter transitions 82 with each diameter transition 82 having an approximately equal length. In another embodiment of the present invention, the gradual change of the diameter from the proximal end 31 to the distal end 24 occurs over a plurality of diameter transitions 82 with each diameter transition 82 having a varying length. The diameter transition 82 refers to a section where the diameter varies from a first diameter to a second diameter.

In a preferred embodiment of the present invention, the length of the ultrasonic probe 15 of the present invention is chosen so as to be resonant in a transverse mode. In an embodiment of the present invention, the ultrasonic probe 15 is between about 30 centimeters and about 300 centimeters in length. In an embodiment of the present invention, the ultrasonic probe 15 is a wire. Those skilled in the art will recognize an ultrasonic probe can have a length shorter than about 30 centimeters and a length longer than about 300 centimeters and be within the spirit and scope of the present invention.

In a preferred embodiment of the present invention, the ultrasonic probe 15 is operated in a transverse mode of operation. The handle 88 surrounds the transducer located between the proximal end 31 of the ultrasonic probe 15 and the connector 93. In a preferred embodiment of the present invention, the transducer includes, but is not limited to, a horn, an electrode, an insulator, a backnut, a washer, a piezo microphone, and a piezo drive. The transducer converts electrical energy provided by the ultrasonic energy source 99 to mechanical energy. The transducer transmits ultrasonic energy received from the ultrasonic energy source 99 to the ultrasonic probe 15. Energy from the ultrasonic energy source 99 is transmitted along the longitudinal axis of the ultrasonic probe 15, causing the ultrasonic probe 15 to vibrate in a transverse mode. The transducer is capable of engaging the ultrasonic probe 15 at the proximal end 31 with sufficient restraint to form an acoustical mass that can propagate the ultrasonic energy provided by the ultrasonic energy source 99.

The ultrasonic energy source 99 produces a transverse ultrasonic vibration along a portion of the longitudinal axis of the ultrasonic probe 15. The ultrasonic probe 15 can support the transverse ultrasonic vibration along the portion of the longitudinal axis of the ultrasonic probe 15. The transverse mode of vibration of the ultrasonic probe 15 according to the present invention differs from an axial (or longitudinal) mode of vibration disclosed in the prior art. Rather than vibrating in an axial direction, the ultrasonic probe 15 of the present invention vibrates in a direction transverse (not parallel) to the axial direction. As a consequence of the transverse vibration of the ultrasonic probe 15, the occlusion destroying effects of the ultrasonic medical device 11 are not limited to those regions of the tip of the ultrasonic probe 15 that may come into contact with an occlusion. Rather, as a section of the longitudinal axis of the ultrasonic probe 15 is positioned in proximity to an occlusion, a diseased area or lesion, the occlusion is removed in all areas adjacent to a plurality of energetic transverse nodes and transverse anti-nodes that are produced along a portion of the longitudinal axis of the ultrasonic probe 15, typically in a region having a radius of up to about 6 mm around the ultrasonic probe 15.

Transversely vibrating ultrasonic probes for occlusion ablation are described in the Assignee's U.S. Pat. No. 6,551,337 and co-pending patent applications U.S. Ser. No. 09/776,015 and U.S. Ser. No. 09/917,471, which further describe the design parameters for such an ultrasonic probe and its use in ultrasonic devices for an ablation, and the entirety of these patents and patent applications are hereby incorporated herein by reference.

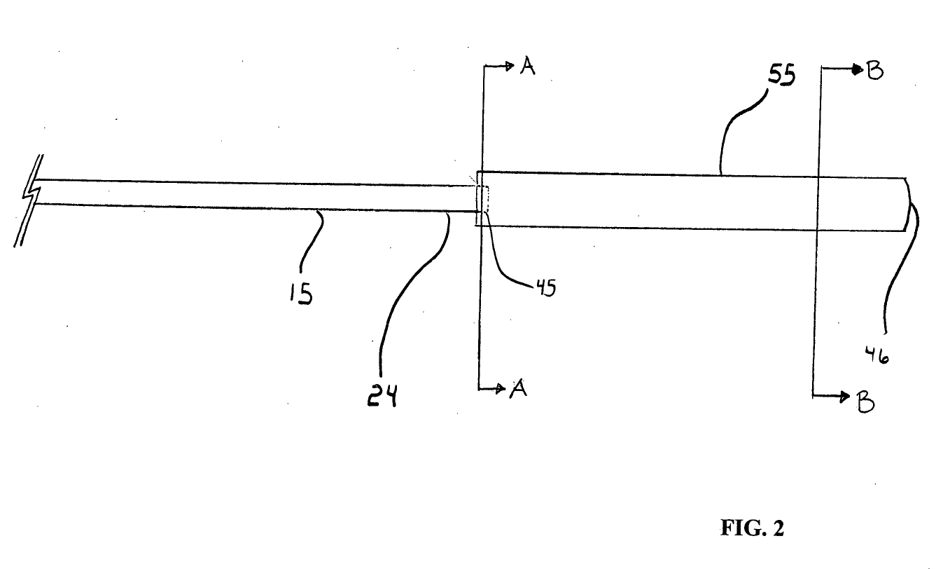

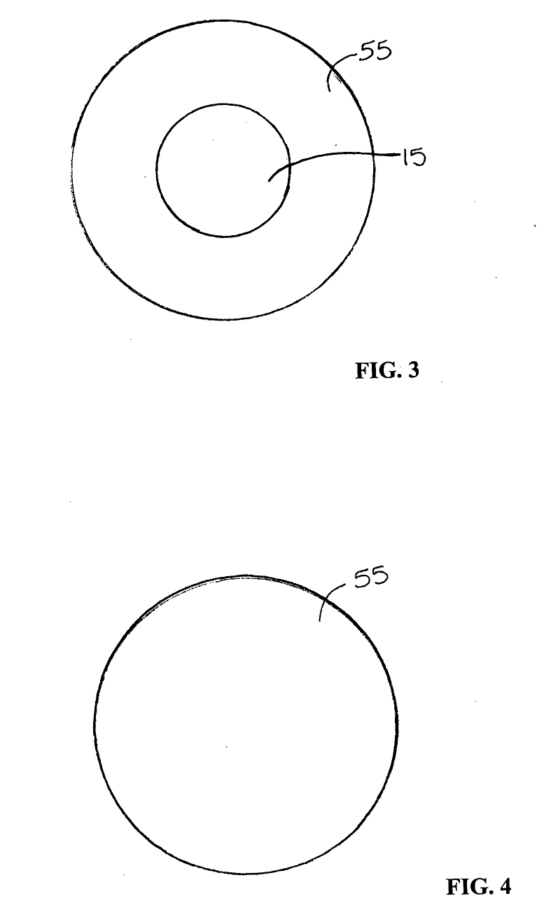

FIG. 2 shows a fragmentary side plan view of the preferred embodiment of the ultrasonic probe 15 of the present invention with a section of a flexible material 55 surrounding the probe tip 9 and extending from the distal end 24 of the ultrasonic probe 15. In this embodiment, the flexible material 55 extends from the probe tip 9 and the flexible material 55 is comprised of a polymer material. The ultrasonic medical device 11 comprises a connecting segment 45 engaging the distal end 24 of the ultrasonic probe 15 and the flexible material 55 extending from the connecting segment 45, the flexible material ending in the flexible material tip 46. FIG. 3 illustrates a cross section of the connecting segment 45, as taken along line A-A of FIG. 2, where the cross section of the connecting segment 45 comprises an inner core of the material comprising the ultrasonic probe and surrounded by the flexible material 55. FIG. 4 illustrates a cross section of the flexible material 55 taken along line B-B of FIG. 2, where the cross section of the flexible material 55 is only the polymer material.

In a preferred embodiment of the present invention, the flexible material 55 is more flexible than the ultrasonic probe 15. In a preferred embodiment of the present invention, the flexible material 55 comprises a polymer material. The polymer material should offer flexibility, impact resistance, very good dynamic properties and resistance to chemical attack. Examples of such polymer materials include, but are not limited to, PEBAX® resin, commercially available from Atofina Chemicals, Inc. of Philadelphia, Pa. (www.atofinachemicals.com). PEBAX® resins are polyether-block co-polyamide polymers that are plasticiser-free thermoplastic elastomers. PEBAX® resins combine the normal ease of processing and properties of the polyamides with the elastomeric qualities of rubbers. Those skilled in the art will recognize the flexible material of the present invention can be comprised of many other materials having similar characteristics known in the art and be within the spirit and scope of the present invention.

In an embodiment of the present invention, the flexible material 55 comprises a mixture of the polymer material and a material of high radiopacity. Materials of high radiopacity do not allow the passage of a substantial amount of x-rays or other radiation. A material of high radiopacity allows a higher degree of visibility in an imaging procedure (such as fluoroscopy, conventional radiography, tomography, digital x-ray imaging, ultrasound, magnetic resonance imaging, and similar procedures) than a material of low radiopacity. The radiopacity of various materials results in radiographs showing different radiopacities so the materials can be differentiated. Radiographic interpretation is based on the visualization and analysis of opacities on a radiograph. As x-ray photons move through the body, the x-ray photons will be attenuated by the tissue and some x-ray photons will pass through the tissue to interact with and expose the radiographic film. The greater the amount of tissue absorption, the fewer the number of x-ray photons reaching the film and the higher the degree of visibility of the material on the radiograph.

Ultrasonic probes utilizing materials of high radiopacity are described in Assignee's co-pending patent applications U.S. Ser. No. 10/328,202 and U.S. Ser. No. 10/207,468, which further describe the design parameters for such an ultrasonic probe and its use in ultrasonic devices for an ablation, and the entirety of these applications are hereby incorporated herein by reference.

The absorption of x-rays is a function of the atomic number and thickness of the material. Materials with a higher atomic number will absorb more radiation than materials with a lower atomic number. The atomic number indicates the internal structure for the atom of each element and the atomic number corresponds to the number of protons in the nucleus of an atom of that element. The atomic number also corresponds to the number of electrons in the neutral atom. The larger the number of electrons floating around the nucleus of a material, the higher the radiopacity is of that material. The mean excitation energy is used in comparing the relative radiopacity of elements. Elements with low radiopacity include hydrogen, helium and titanium. Hydrogen (atomic number of 1) has a mean excitation energy of 19.2 electron-volts and helium (atomic number of 2) has a mean excitation energy of 41.8 electron-volts. Titanium (atomic number of 22) has a mean excitation energy of 233 electron-volts. Materials with high radiopacity include, uranium, lead, gold, tantalum and tungsten. Uranium (atomic number of 92) has a mean excitation energy of 890 electron-volts, lead (atomic number of 82) has a mean excitation energy of 823 electron-volts and gold (atomic number of 79) has a mean excitation energy of 790 electron-volts. Tantalum (atomic number of 73) has a mean excitation energy of 718 electron-volts and tungsten (atomic number of 74) has a mean excitation energy of 727 electron-volts. Other materials of high radiopacity that could be used within the spirit and scope of the present invention include barium sulfate, molybdenum and alloys thereof. Those skilled in the art will recognize that other materials of high radiopacity known in the art would be within the spirit and scope of the present invention.

In another embodiment of the present invention, the flexible material 55 comprises the polymer material with a coating of the high radiopacity material. The high radiopacity coating is applied in manners known in the art including, but not limited to, pad printing, molding, silk screening, direct application and similar processes. Those skilled in the art will recognize the flexible material can comprise the material of high radiopacity in many ways known in the art and be within the spirit and scope of the present invention.

In an embodiment of the present invention, the flexible material 55 surrounds the distal end 24 of the ultrasonic probe 15. The flexible material 55 protects a vasculature as the ultrasonic probe 15 is moved through the vasculature. The vasculature in the body can follow tortuous paths where the ultrasonic probe 15 contacts the wall of the vasculature as the ultrasonic probe 15 is navigated along the vasculature. The flexible material 55 cushions at least a portion of the longitudinal axis of the ultrasonic probe 15 and the probe tip 9 as the ultrasonic probe 15 is moved through the vasculature. In addition to cushioning the probe tip 9, the flexible material 55 reduces the stresses on the ultrasonic probe 15 as the ultrasonic probe 15 is navigated within the vasculature. The flexible material 55 absorbs some of the contact stresses and lessens contact stresses imparted to the ultrasonic probe 15, thereby preserving the ultrasonic and mechanical properties of the ultrasonic probe 15. Without the flexible material 55, the ultrasonic probe 15 is more susceptible to changes in its acoustic behavior while in contact with the vasculature.

The present invention also provides a method for applying a flexible material to an ultrasonic medical device. One of the primary challenges in applying a polymer coating or overmold to the ultrasonic medical device is the process of applying the polymer to the medical device, i.e., a titanium ultrasonic probe. The small diameter and flexibility of the ultrasonic probe cause difficulty in applying the required amount of polymer uniformly to the probe. Furthermore, the polymer must adhere in such a way to withstand the acoustic vibrations of the probe. The polymer may be overmolded to the ultrasonic probe through an injection molding process. However, the pressure required to inject the polymer into a mold may cause the probe to bend, kink, or be off-centered which would damage the ultrasonic device, as well as cause non-uniform application of the polymer. Furthermore, other complications may occur such as flash and non-uniformity due to the small channel through which the polymer must be injected. As such, there is a need in the art for a process of applying a polymer to an ultrasonic medical device wherein the polymer enhances the flexibility, tip softness, radiopacity and acoustic properties of the ultrasonic probe. In addition, the flexible material engaged to the ultrasonic medical device must be able to withstand the vibrations of the medical device.

The present invention teaches a method of overcoming these challenges by a novel process of applying a flexible material to an ultrasonic medical device. While the process of the present invention may be used for applying polymers to ultrasonic devices, it is not limited to ultrasonic devices. Those skilled in the art will recognize that various instruments and/or devices may be engaged to various materials and the process will remain within the spirit and scope of the invention.

The present invention provides a method for adhering a flexible material to an ultrasonic medical device comprising: providing the flexible material to be adhered to the ultrasonic medical device; engaging the flexible material to the ultrasonic medical device; heating the flexible material engaged to the ultrasonic medical device with a heat source causing the flexible material to melt; and cooling the flexible material engaged to the ultrasonic medical device.

In an embodiment, the flexible material is a polymer. The polymer is pre-extruded into a desired shape and size. The optimal shape of the pre-extruded polymer comprises a hollow channel through the polymer (i.e., a tube) to allow the ultrasonic probe to be inserted into the polymer. The polymer to be used will be selected for optimal adhesion to the ultrasonic probe. Additionally, the polymer may comprise a material of high radiopacity. In another embodiment, the polymer is injection molded. In another embodiment, the polymer is dip coated. Those skilled in the art will recognize that various methods of forming thermal plastic polymers are within the spirit and scope of the present invention.

The ultrasonic probe is then advanced through the polymer. The ultrasonic probe may be advanced manually or by a machine. The polymer and the ultrasonic probe are placed within an oven. Once in the oven, the polymer is heated above its melting point. Such heating allows the polymer to melt and adhere to the ultrasonic probe. Depending on the temperature of the oven, the heating time may vary. On average, the heating time is approximately 3-5 minutes. In an alternative embodiment, the heat source is a hot air system that allows for quick heating and cooling of the mold. In another embodiment, the heat source may be a heating block. Those skilled in the art will recognize that various heat sources and various heating times may be used and still be with in the spirit and scope of the present invention.

Following the heating step, the polymer is then cooled to return to its solid state, resulting in the polymer adhering to the ultrasonic probe. Those of skill in the art will recognize that additional steps may be added to the process and still fall within the spirit and scope of the present invention.

In an alternative embodiment of the present invention, a polymer may be provided as a solid extrusion, i.e., a prepared polymer that does not comprise a hollow channel. In accordance with this embodiment, the polymer is allowed to melt within a mold before the introduction of the ultrasonic medical device. Once the polymer has melted due to the addition of heat, the ultrasonic device is introduced into the mold. Following the addition of the ultrasonic probe, the polymer is then cooled to return to its solid state, resulting in the polymer adhering to the ultrasonic probe.

In an alternative embodiment, a heat shrink may be used over the polymer to enhance the adhesion of the polymer to the ultrasonic probe. A heat shrink comprises a polymer layer which is applied over the flexible material engaged to the ultrasonic probe. The heat shrink adds another mechanism of fastening the polymer to the ultrasonic probe. A heat shrink increases the safety of the ultrasonic device. The heat shrink also prevents particulate from releasing from the polymer. Additionally, a heat shrink reduces the possibility of air gaps within the polymer which can lead to failure during vibration. In one embodiment of the present invention, the heat shrink is applied in an expanded state prior to melting the polymer. The heat shrink is in the expanded state prior to heating the heat shrink. Once heated, the polymer layer comprising the heat shrink will shrink and compress the flexible material against the ultrasonic probe. In another embodiment, the heat shrink is applied after the polymer is adhered to the ultrasonic probe. In one embodiment of the present invention, the heat shrink comprises polyolefin. Those skilled in the art will recognize that various materials may comprise the heat shrink and remain within the spirit and scope of the invention.

In an embodiment of the present invention, the flexible material 55 engages the longitudinal axis of the ultrasonic probe 15 by a process of melt forming. Melt forming is a process that reforms the polymer by heating the polymer to the melt phase and allowing the material to flow evenly into the desired shape. The melt forming process ensures an even distribution of materials through the entire cross section of the shaped object and adhesion to the probe. Those skilled in the art will recognize that additional steps may be added to the melt forming process and still be within the spirit and scope of the present invention.

In another embodiment of the present invention, the flexible material 55 engages at least a portion of the longitudinal axis of the ultrasonic probe 15 by a process of joining or attaching via heat shrink tubing or the like. Heat shrink tubing can be slipped over the flexible material and probe. When heat is applied the tubing will shrink down and create a compressive force to hold the materials together. Those skilled in the art will recognize that additional steps may be added to the heat shrink process and still be within the spirit and scope of the present invention. Shrink fitting typically refers to the joining of metals via a heating process that changes the dimensions of the metal.

In another embodiment of the present invention, the flexible material 55 is overmolded along at least a portion of the longitudinal axis of the ultrasonic probe 15 by a process of injection molding. Injection molding involves taking plastic in the form of pellets or granules and heating this material until a melt is obtained. The melt is injected into a split die chamber or mold where it cools into the desired shape on the probe. The mold is then opened and the part is ejected. Those skilled in the art will recognize that additional steps may be added to the injection molding process and still be within the spirit and scope of the present invention.

In another embodiment of the present invention, the flexible material 55 engages at least a portion of the longitudinal axis of the ultrasonic probe by a process of dip coating. Dip coating involves the heating of an object onto which the polymer is to be coated. The heated parts are immersed in a tank of a polymer material, where heat from the part attracts the polymer material and the assembly is formed. The parts are extracted from the liquid and heat cured. Those skilled in the art will recognize that additional steps may be added to the dip coating process and still be within the spirit and scope of the present invention.

In another embodiment of the present invention, the flexible material 55 engages at least a portion of the longitudinal axis of the ultrasonic probe with an adhesive. In a preferred embodiment of the present invention, the adhesive is cyanoacryalate. Cyanoacrylate is a one component adhesive that cures within about 1 minute to about 3 minutes through moisture absorption. Cyanoacrylate adhesives permit bonding of many different materials, providing bonds of high strength and high resistance. Those skilled in the art will recognize that additional steps may be added to the adhesion process and still be within the spirit and scope of the present invention.

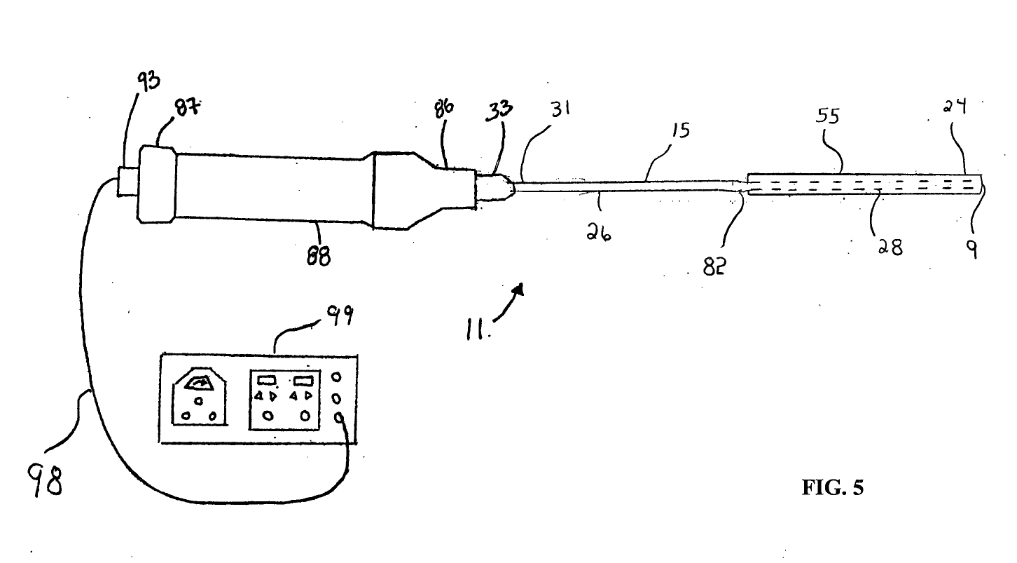

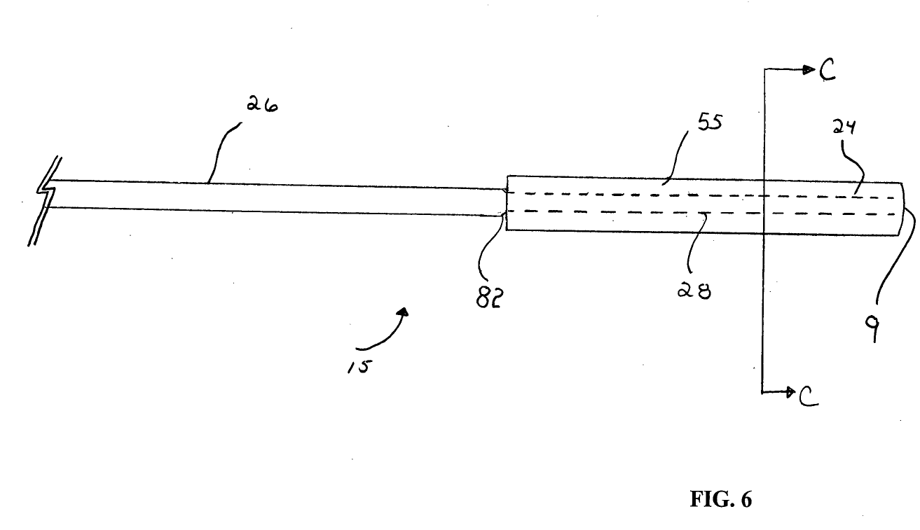

As shown in FIG. 5, an alternative embodiment of the present invention comprises a flexible material 55 surrounding a portion of a longitudinal axis of an ultrasonic probe 15. In one embodiment, the flexible material ends at the probe tip 9. In another embodiment, the flexible material 55 extends beyond the probe tip 9. Those skilled in the art will recognize the flexible material can span from any point along the longitudinal axis of the ultrasonic probe and span to any point along the longitudinal axis of the ultrasonic probe or beyond the distal end of the ultrasonic probe and be within the spirit and scope of the present invention.

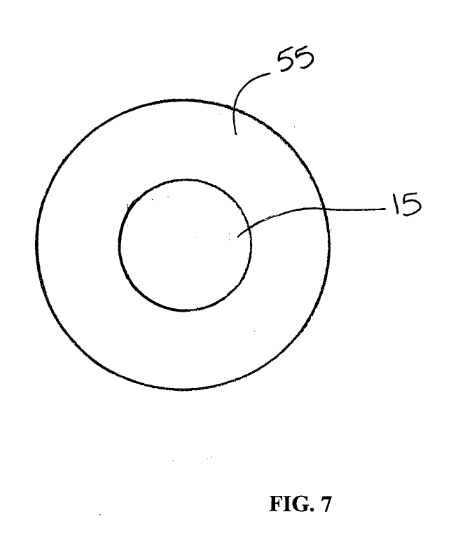

FIG. 6 shows a fragmentary side plan view of an alternative embodiment of the present invention with a portion of a longitudinal axis of the ultrasonic probe 15 surrounded by the flexible material 55. In one embodiment of the present invention, the flexible material 55 extends beyond the probe tip 9. In one embodiment of the present invention, the flexible material 55 ends at the probe tip 9. FIG. 7 shows a cross section taken along line C-C of FIG. 6, illustrating the ultrasonic probe 15 surrounded by the flexible material 55.

In a preferred embodiment of the present invention, the diameter of the portion of the longitudinal axis that includes the flexible material 55 is about 0.014 inches. In another embodiment of the present invention, the diameter of the portion of the longitudinal axis that includes the flexible material 55 is smaller than about 0.014 inches. In another embodiment of the present invention, the diameter of the portion of the longitudinal axis that includes the flexible material 55 is larger than about 0.014 inches. Those skilled in the art will recognize the diameter of the portion of the longitudinal axis that includes the flexible material can have a varying diameter and be within the spirit and scope of the present invention.

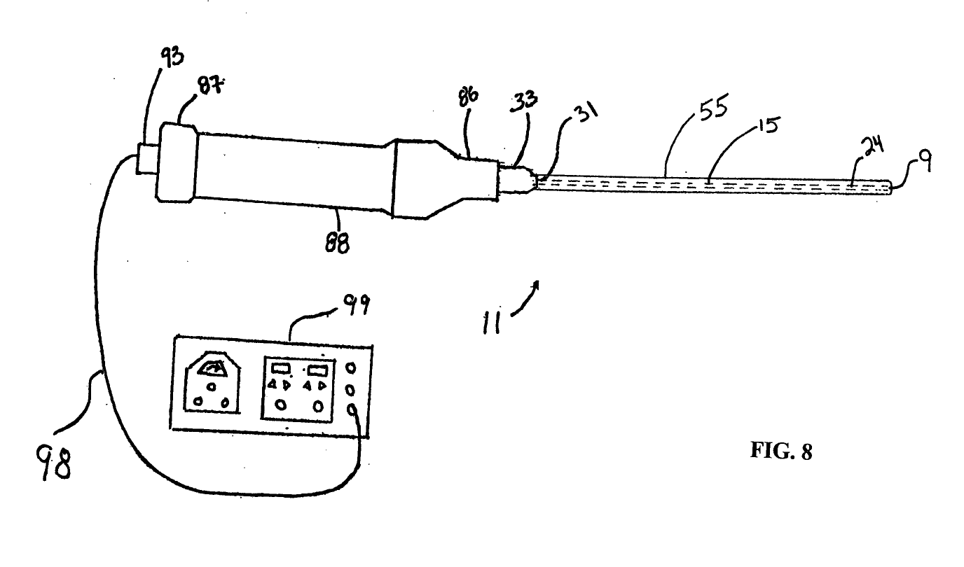

FIG. 8 shows an alternative embodiment of the present invention wherein the longitudinal axis of an ultrasonic probe 15 is surrounded by a flexible material 55 from the proximal end 31 to the distal end 24. In one embodiment, the flexible material 55 ends at the probe tip 9. In another embodiment, the flexible material 55 extends beyond the probe tip 9. Those skilled in the art will recognize that the flexible material 55 may begin and end at various positions along the longitudinal axis of the ultrasonic probe 15 and still be within the spirit and scope of the invention.

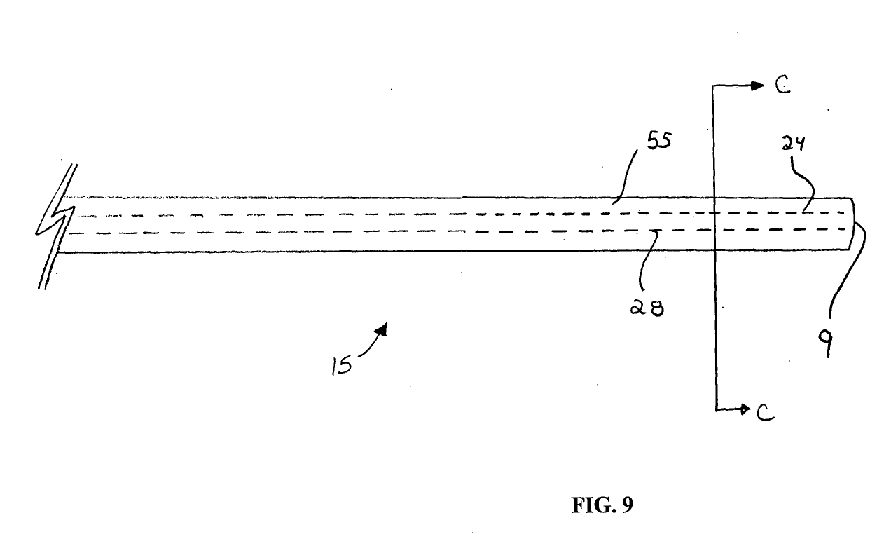

FIG. 9 shows a fragmentary side plan view of the ultrasonic probe 15 of the present invention shown in FIG. 8 with a longitudinal axis of the ultrasonic probe 15 surrounded by a flexible material 55. FIG. 7 shows a cross section taken along line C-C of FIG. 9, illustrating the ultrasonic probe 15 surrounded by the flexible material 55.

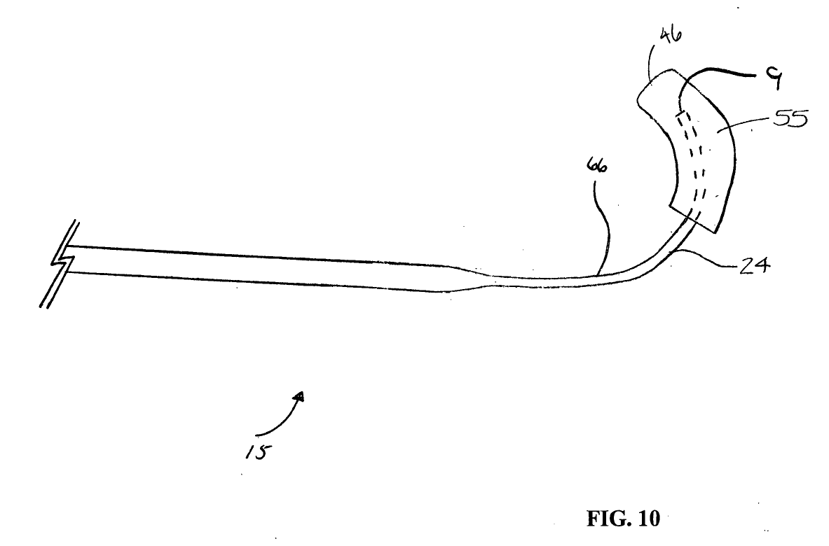

FIG. 10 shows a fragmentary side plan view of an alternative embodiment of the ultrasonic probe 15 of the present invention with the portion of the longitudinal axis of the ultrasonic probe 15 surrounded by the flexible material 55 having a curved shape. The flexible material 55 surrounds a portion of the longitudinal axis of the ultrasonic probe including the probe tip 9. The flexible material 55 extends beyond the probe tip 9 and ends at the flexible material tip 46. In another embodiment of the invention, the flexible material 55 ends at the probe tip 9. A portion of the longitudinal axis of the ultrasonic probe 15 and the flexible material 55 deviates from a straight length at a shape transition 66. The curved shape of the distal end 24 of the ultrasonic probe 15 and the flexible material 55 is beneficial when the site of the occlusion is at a sharp bend in the vasculature. The non-linear shape allows for treatment of occlusions or steerability to reach the occlusions when traversing through a tortuous anatomy which could not be reached by a linear device because the non-linear shape increases flexibility and maneuverability of the ultrasonic probe 15.

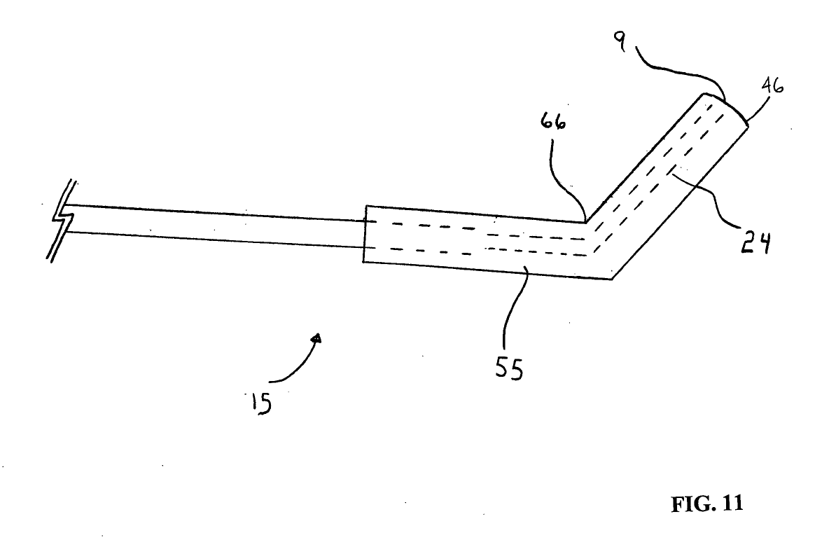

FIG. 11 shows a fragmentary side plan view of an alternative embodiment of the ultrasonic probe 15 of the present invention with the portion of the longitudinal axis of the ultrasonic probe 15 surrounded by the flexible material 55 at an angle to the longitudinal axis of the ultrasonic probe 15. The flexible material 55 surrounds a portion of the longitudinal axis of the ultrasonic probe 15 including the probe tip 9. The flexible material 55 extends beyond the probe tip 9 and ends in the flexible material tip 46. A section of the portion of the longitudinal axis of the ultrasonic probe 15 surrounded by the flexible material 55 deviates from a straight length at the shape transition 66. The angled shape of the distal end 24 of the ultrasonic probe 15 and the flexible material 55 is beneficial when the site of the occlusion 16 is at a sharp bend in the vasculature 44. Those skilled in the art will recognize that other curved or bent shapes of the ultrasonic probe are within the spirit and scope of the invention.

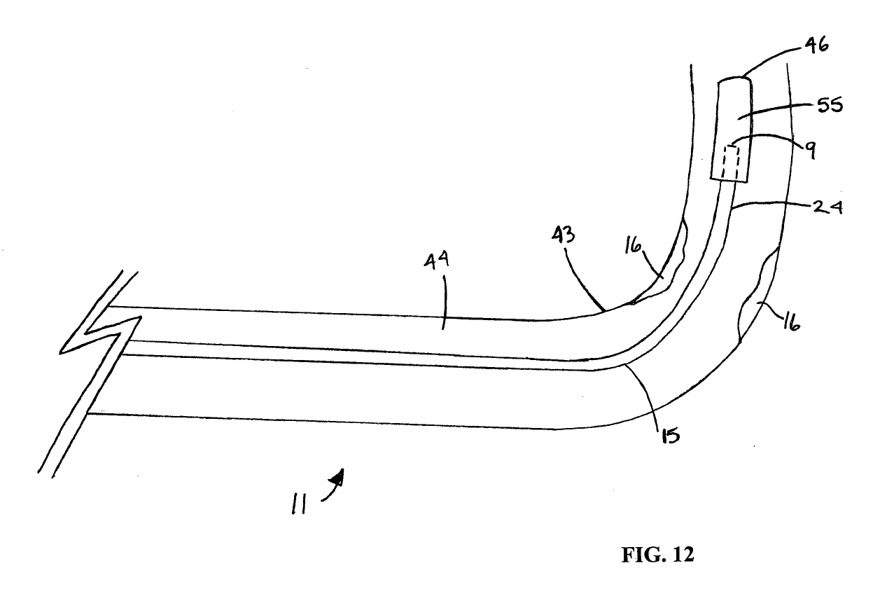

FIG. 12 shows a fragmentary side plan view of the ultrasonic probe 15 of the present invention with the longitudinal axis of the ultrasonic probe 15 located at a bend 43 in the vasculature 44 and proximal to an occlusion 16 wherein the probe tip 9 is surrounded by the flexible material 55 that extends beyond the probe tip 9. In a preferred embodiment of the present invention, the occlusion 16 comprises a biological material. FIG. 12 illustrates a general working area for the ultrasonic medical device 11 in the vasculature 44.

The flexible material 55 facilitates navigation of the ultrasonic medical device 11 within the vasculature 44. The flexible material 55 improves a trackability of the ultrasonic probe 15 through the vasculature 44. By providing the flexible material 55 and shaping the section of the portion of the longitudinal axis of the ultrasonic probe 15 surrounded by the flexible material 55 at the distal end 24 of the ultrasonic probe 15, the ultrasonic probe 15 is moved along the tortuous paths of the vasculature 44 in an easy and safe manner. By shaping the section of the portion of the longitudinal axis of the ultrasonic probe 15 with the flexible material 55, a radial span of the ultrasonic medical device 11 is increased as a plurality of points of the ultrasonic probe 15 are placed closer to the occlusion 16. The flexible material 55 provides the flexibility that allows the ultrasonic probe 15 to be maneuvered along the bend 43 in the vasculature 44 to place the ultrasonic probe 15 in closer proximity to the occlusion 16. Because the flexible material 55 provides the flexibility to allow the ultrasonic probe 15 to be moved along the bend 43 in the vasculature 44, the surface area of the ultrasonic probe 15 in communication with the occlusion is increased.

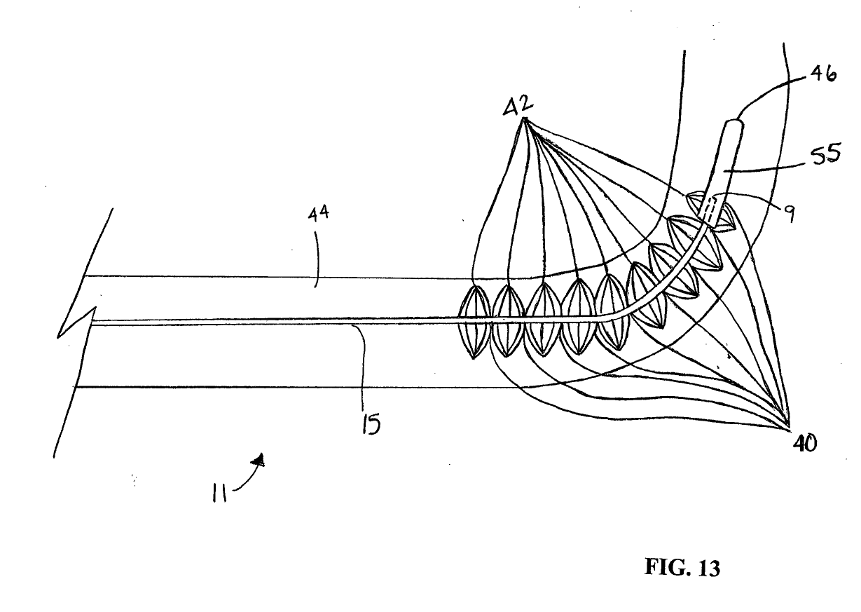

FIG. 13 shows a fragmentary side plan view of the ultrasonic probe 15 of the present invention located at a bend in the vasculature 44 showing a plurality of transverse nodes 40 and a plurality of transverse anti-nodes 42 along a portion of the longitudinal axis of the ultrasonic probe 15. The transverse nodes 40 are areas of minimum energy and minimum vibration. A plurality of transverse anti-nodes 42, areas of maximum energy and maximum vibration, also occur at repeating intervals along the portion of the longitudinal axis of the ultrasonic probe 15. The number and spacing of transverse nodes 40 and transverse anti-nodes 42 of the ultrasonic probe 15 depend on the frequency of energy produced by the ultrasonic energy source 99. As shown in FIG. 13, the flexible material 55 engaging a portion of the longitudinal axis of the ultrasonic probe 15 dampens the amplitude of vibration over the portion of the ultrasonic probe 15 surrounded by the flexible material 55. Due to the dampened amplitude of vibration, a smaller amount of energy is transferred. As such, the occlusion destroying effect of the ultrasonic probe 15 is greater along the portion of the ultrasonic probe 15 not surrounded by the flexible material than over the portion of the ultrasonic probe 15 surrounded by the flexible material 55.

By providing the flexibility that allows the ultrasonic probe 15 to be bent around the vasculature 44 and shaped, the flexible material 55 allows a treatment area of the ultrasonic probe 15 to be expanded as the ultrasonic probe 15 is positioned closer to the occlusion 16, enabling a larger active area of the ultrasonic probe 15 to be in communication with the occlusion 16 when compared to a probe that is approximately straight along the longitudinal axis. The plurality of transverse nodes 40 and transverse anti-nodes 42 produced by the transverse ultrasonic vibration produces an occlusion destroying effect in a region around the longitudinal axis of the ultrasonic probe 15. Since the occlusion destroying effects of the ultrasonic probe 15 are in a region having a radius of up to about 6 mm around the longitudinal axis of the ultrasonic probe 15, the shaped segment of the ultrasonic probe 15 allows the occlusion destroying effects of the ultrasonic probe 15 to cover a larger radial span of the vasculature 44 to ablate the occlusion 16.

The ultrasonic energy source 99 provides a low power electric signal of approximately 2 watts to the transducer that is located within the handle 88. The transducer converts electrical energy provided by the ultrasonic energy source 99 to mechanical energy. Piezoelectric ceramic crystals inside the transducer create an axial motion that is converted into a standing transverse wave along the longitudinal axis of the ultrasonic probe 15. In a preferred embodiment of the present invention, the transducer is a piezoelectric transducer that is coupled to the ultrasonic probe 15 to enable transfer of ultrasonic excitation energy and cause the ultrasonic probe 15 to oscillate in a transverse direction relative to the longitudinal axis. In an alternative embodiment of the present invention, a magneto-strictive transducer may be used for transmission of the ultrasonic energy.

Through a process of cavitation, the transverse wave generates acoustic energy in the surrounding fluid. Cavitation is a process in which small voids are formed in a surrounding fluid through the rapid motion of the ultrasonic probe 15 and the voids are subsequently forced to compress. The compression of the voids creates a wave of acoustic energy which acts to dissolve the matrix binding together the occlusion 16, while having no damaging effects on healthy tissue.

Because the shaped segment of the ultrasonic probe 15 has a plurality of points along the longitudinal axis positioned closer to the occlusion 16, the power required to vibrate the longitudinal axis of the ultrasonic probe 15 and ablate the occlusion 16 can be minimized. High power levels can adversely affect the vasculature 44 and the patient because high power levels provide a shock effect. In addition, since the shaped segment is closer to the occlusion 16, the treatment time to remove the occlusion 16 is minimized. Long treatment times for the ablation of the occlusion 16 are undesirable as the vasculature 44 becomes more susceptible to potential damage the longer the ultrasonic probe 15 is inserted into the vasculature 44.

The extent of the acoustic energy produced from the ultrasonic probe 15 is such that the acoustic energy extends radially outward from the longitudinal axis of the ultrasonic probe 15 at the transverse anti-nodes 42 along the portion of the longitudinal axis of the ultrasonic probe 15. In this way, actual treatment time using the transverse mode ultrasonic medical device 11 according to the present invention is greatly reduced as compared to methods disclosed in the prior art that primarily utilize longitudinal vibration (along the axis of the ultrasonic probe). A distinguishing feature of the present invention is the ability to utilize ultrasonic probes 15 of extremely small diameter compared to prior art probes.

The number of transverse nodes 40 and transverse anti-nodes 42 occurring along the longitudinal axis of the ultrasonic probe 15 is modulated by changing the frequency of energy supplied by the ultrasonic energy source 99. The exact frequency, however, is not critical and the ultrasonic energy source 99 run at, for example, about 20 kHz is sufficient to create an effective number of occlusion 16 destroying transverse anti-nodes 42 along the longitudinal axis of the ultrasonic probe 15. The low frequency requirement of the present invention is a further advantage in that the low frequency requirement leads to less damage to healthy tissue. Those skilled in the art understand it is possible to adjust the dimensions of the ultrasonic probe 15, including diameter, length and distance to the ultrasonic energy source 99, in order to affect the number and spacing of the transverse nodes 40 and transverse anti-nodes 42 along a portion of the longitudinal axis of the ultrasonic probe 15.

The present invention allows the use of ultrasonic energy to be applied to the occlusion 16 selectively, because the ultrasonic probe 15 conducts energy across a frequency range from about 10 kHz through about 100 kHz. The amount of ultrasonic energy to be applied to a particular treatment site is a function of the amplitude and frequency of vibration of the ultrasonic probe 15. In general, the amplitude or throw rate of the energy is in the range of about 25 microns to about 250 microns, and the frequency in the range of about 10 kHz to about 100 kHz. In a preferred embodiment of the present invention, the frequency of ultrasonic energy is from about 20 kHz to about 35 kHz. Frequencies in this range are specifically destructive of occlusions 16 including, but not limited to, hydrated (water-laden) tissues such as endothelial tissues, while substantially ineffective toward high-collagen connective tissue, or other fibrous tissues including, but not limited to, vascular tissues, epidermal, or muscle tissues.

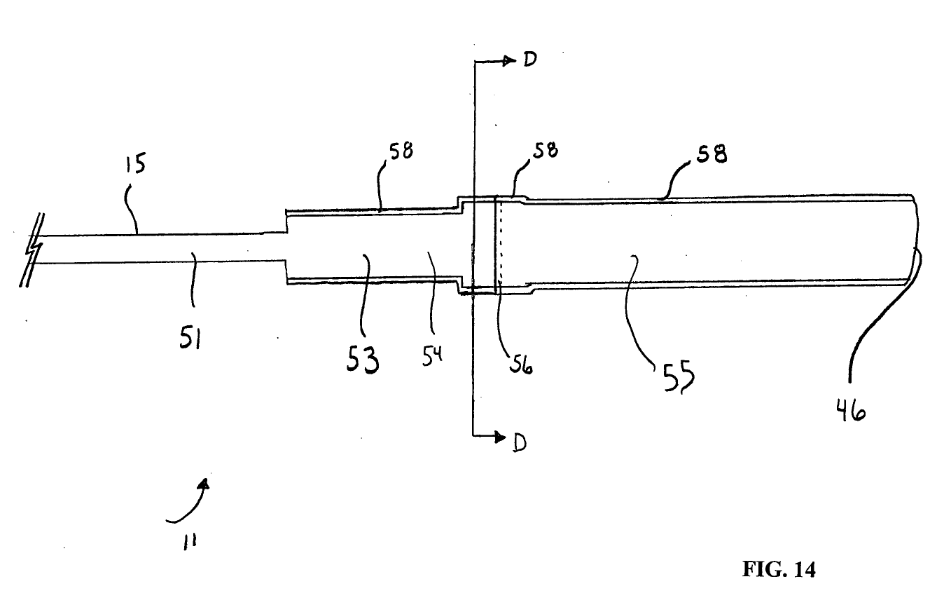

FIG. 14 shows a fragmentary side plan view of an alternative embodiment of the ultrasonic probe 15 of the present invention with a section of the ultrasonic probe 15 and a portion of a longitudinal axis of the flexible material 55 surrounded by a heat shrink 58. As shown in FIG. 14, a thin layer of heat shrink may be used to engage the flexible material 55 to the ultrasonic probe 15. In the alternative embodiment of the present invention shown in FIG. 14, the diameter of the ultrasonic probe 15 increases in a stepwise fashion or similar near the distal end 24 of the ultrasonic probe 15. The increase in diameter near the distal end 24 of the ultrasonic probe 15 allows for the engagement of the flexible material 55 to the ultrasonic probe 15. The ultrasonic probe 15 includes intervals 51, 53 and 54 where the diameter of the ultrasonic probe 15 increases from interval 51 to interval 53 to interval 54.

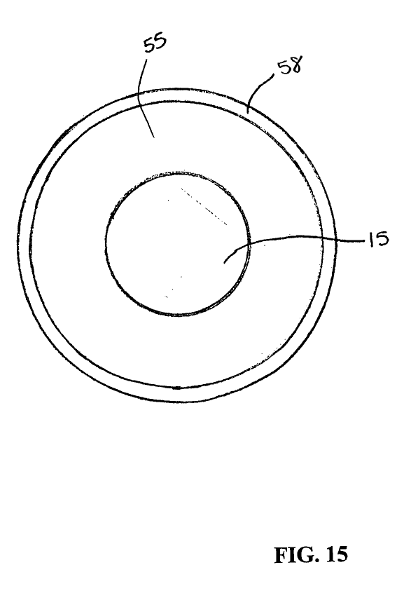

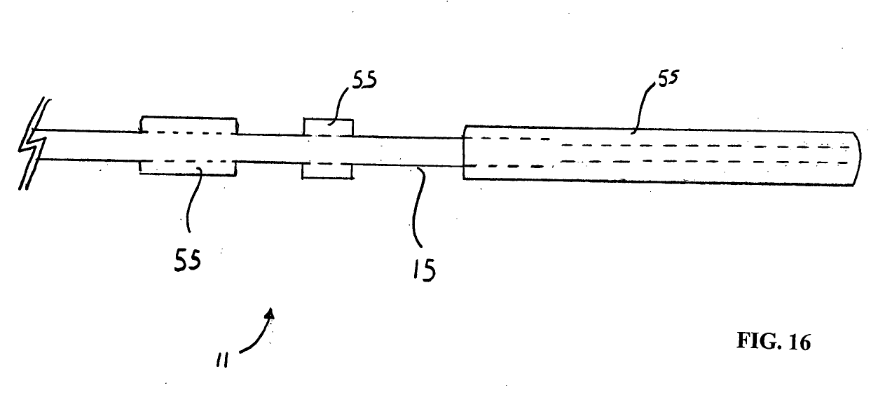

FIG. 15 shows a cross section view of the connecting segment of the ultrasonic probe 15 taken along line D-D of FIG. 14, where the segment has a cross section having a core of the ultrasonic probe 15, surrounded by the flexible material 55 which is further surrounded by the heat shrink 58. FIG. 16 shows a fragmentary side plan view of the ultrasonic probe 15 of an alternative embodiment of the present invention with a flexible material 55 along a plurality of locations of the longitudinal axis of the ultrasonic probe 15. An ultrasonic probe 15 having a plurality of locations engaging the flexible materials 55 along the longitudinal axis protects the vasculature 44 as the ultrasonic probe 15 is moved along the vasculature 44 and cushions the ultrasonic probe 15 to prevent harm to the ultrasonic probe 15, the vasculature 44 and the patient, or provides a radiopaque marker. Those skilled in the art will recognize the ultrasonic probe 15 can have any flexible materials along the longitudinal axis of the ultrasonic probe 15 and still be within the spirit and scope of the present invention.

The present invention provides a method of moving the ultrasonic probe 15 along a path in the vasculature 44 of the body to remove an occlusion 16. A flexible material 55 engages the ultrasonic probe 15 and the ultrasonic probe 15 with the flexible material 55 is inserted into the vasculature 44. The ultrasonic probe 15 is advanced along the vasculature 44 until the flexible material 55 contacts a wall of the vasculature 44 to allow the ultrasonic probe 15 to bend along the path in the vasculature 44. The ultrasonic probe 15 is moved further along the vasculature 44 and positioned in proximity to the occlusion 16.

As discussed above, the flexible material 55 may engage the ultrasonic probe 15 by processes including, but not limited to, pad printing, injection molding, melt forming, overmolding, silk screening, direct application and similar processes. Those skilled in the art will recognize the flexible material 55 can engage the ultrasonic probe 15 in many ways known in the art and be within the spirit and scope of the present invention.

The present invention also provides a method of ablating an occlusion 16. In a preferred embodiment of the present invention, the occlusion 16 comprises a biological material. A flexible material 55 engages the ultrasonic probe 15 at an at least one location of the ultrasonic probe 15. At least one location of the ultrasonic probe 15 with the flexible material 55 may be shaped to increase a radial span of the ultrasonic probe 15. The ultrasonic probe 15 with the flexible material 55 is inserted into the vasculature 44 of the body and advanced within the vasculature 44. An ultrasonic energy source is activated to provide an ultrasonic energy to the ultrasonic probe 15 to ablate the occlusion 16.

The present invention provides a method of protecting the vasculature 44 and preserving the ultrasonic and mechanical properties of the ultrasonic probe 15. The flexible material 55 cushions a tip 9 of the ultrasonic probe 15 as the ultrasonic probe 15 is moved through the vasculature 44. The flexible material 55 reduces the stresses on the ultrasonic probe 15 as the ultrasonic probe 15 is navigated within the vasculature 44.

The present invention provides an apparatus and a method for an ultrasonic probe engaging a flexible material. The flexible material provides the flexibility to move the ultrasonic probe in the vasculature of the body to remove an occlusion while protecting the vasculature without adversely affecting the functionality of the ultrasonic probe. The present invention provides an ultrasonic probe with a flexible material that is simple, user-friendly, reliable and cost effective.

All patents, patent applications, and published references cited herein are hereby incorporated herein by reference in their entirety. While this invention has been particularly shown and described with references to preferred embodiments thereof, it will be understood by those skilled in the art that various changes in form and details may be made therein without departing from the scope of the invention encompassed by the appended claims.

1. An ultrasonic medical device comprising:
an ultrasonic probe having a proximal end, a distal end and a longitudinal axis therebetween; and
a flexible material engaging the ultrasonic probe, wherein the portion of the longitudinal axis of the ultrasonic probe with the flexible material protects a vasculature as the ultrasonic probe is moved through the vasculature.

2. The device of claim 1 wherein the portion of the longitudinal axis of the ultrasonic probe with the flexible material is shaped to increase a radial span of the ultrasonic medical device.

3. The device of claim 1 wherein the flexible material surrounds the distal end of the ultrasonic probe.

4. The device of claim 1 wherein the portion of the longitudinal axis of the ultrasonic probe with the flexible material is curved.

5. The device of claim 1 wherein the flexible material cushions a tip of the ultrasonic probe as the ultrasonic probe is moved through the vasculature.

6. The device of claim 1 wherein the flexible material facilitates navigation of the ultrasonic medical device within the vasculature.

7. The device of claim 1 wherein the flexible material reduces the stresses on the ultrasonic probe as the ultrasonic probe is navigated within the vasculature.

8. The device of claim 1 wherein the flexible material comprises a material of high radiopacity.

9. The device of claim 8 wherein the material of high radiopacity is tungsten.

10. The device of claim 1 wherein the flexible material comprises a polymer material.

11. The device of claim 1 wherein the ultrasonic probe is a wire.

12. The device of claim 1 wherein the flexible material is more flexible than the ultrasonic probe.

13. The device of claim 1 wherein the distal end of the ultrasonic probe is thinner than the proximal end of the ultrasonic probe.

14. The device of claim 1 wherein the flexible material is melt formed to the ultrasonic probe.

15. The device of claim 1 wherein a layer of shrink fitting is applied to the flexible material and the ultrasonic probe.

16. The device of claim 1 wherein the flexible material is dip molded to the ultrasonic probe.

17. The device of claim 1 wherein the flexible material is injection molded to the ultrasonic probe.

18. The device of claim 1 wherein the flexible material is engaged to the ultrasonic probe at an ultrasonic probe tip.

19. The device of claim 1 wherein the flexible material extends beyond an ultrasonic probe tip.

20. The device of claim 1 wherein the flexible material surrounds the ultrasonic probe from the proximal end of the probe to the distal end of the probe.

21. The device of claim 1 wherein the flexible material surrounds substantially the entire longitudinal axis of the ultrasonic probe.

22. An ultrasonic medical device for removing a biological material comprising:
an elongated ultrasonic probe having a proximal end, a distal end and a longitudinal axis therebetween; and
a flexible material engaging the ultrasonic probe,
wherein the flexible material comprises a material of high radiopacity.

23. The device of claim 22 wherein the flexible material protects a vasculature as the elongated ultrasonic probe is moved through the vasculature.

24. The device of claim 22 wherein the flexible material cushions a tip of the elongated ultrasonic probe as the elongated ultrasonic probe is moved through a vasculature.

25. The device of claim 22 wherein the flexible material improves a trackability of the elongated ultrasonic probe through a vasculature.

26. The device of claim 22 wherein the flexible material reduces the stresses on the elongated ultrasonic probe as the elongated ultrasonic probe is navigated within a vasculature.

27. The device of claim 22 wherein the flexible material comprises a polymer material.

28. The device of claim 22 wherein the flexible material is shaped to increase a radial span of the elongated ultrasonic probe within a vasculature.

29. The device of claim 22 wherein the flexible material is engaged to the ultrasonic probe at an ultrasonic probe tip.

30. The device of claim 22 wherein the flexible material extends beyond an ultrasonic probe tip.

31. The device of claim 22 wherein the flexible material surrounds the ultrasonic probe from the proximal end of the probe to the distal end of the probe.

32. The device of claim 22 wherein the flexible material surrounds substantially the entire longitudinal axis of the ultrasonic probe.

33. A method of moving an ultrasonic probe along a path in a vasculature of a body to remove a biological material comprising:
engaging a flexible material to the ultrasonic probe;
inserting the ultrasonic probe with the flexible material into the vasculature;
advancing the ultrasonic probe in the vasculature until the flexible material contacts a wall of the vasculature to allow the ultrasonic probe to bend along the path in the vasculature; and
moving the ultrasonic probe further along the vasculature.

34. The method of claim 33 wherein the flexible material surrounds at least a portion of a longitudinal axis of the ultrasonic probe.

35. The method of claim 33 wherein the flexible material extends from a distal end of the ultrasonic probe.

36. The method of claim 33 further comprising melt forming the flexible material to the ultrasonic probe.

37. The method of claim 33 further comprising shrink fitting the flexible material to the ultrasonic probe.

38. The method of claim 33 further comprising dip molding the flexible material to the ultrasonic probe.

39. The method of claim 33 further comprising injection molding the flexible material to the ultrasonic probe.

40. The method of claim 33 further comprising engaging the flexible material to the ultrasonic probe with an adhesive.

41. The method of claim 33 wherein the flexible material reduces the stresses on the ultrasonic probe as the ultrasonic probe is moved along the tortuous path in the vasculature.

42. The method of claim 33 wherein the flexible material comprises a material of high radiopacity.

43. The method of claim 33 wherein the flexible material is shaped to facilitate navigation within the vasculature.

44. The method of claim 33 further comprising shaping the flexible material to increase a radial span of the ultrasonic medical device within the vasculature.

45. The method of claim 33 wherein the flexible material protects the vasculature as the ultrasonic probe is moved through the vasculature.

46. The method of claim 33 wherein the flexible material cushions a tip of the ultrasonic probe as the ultrasonic probe is moved through the vasculature.

47. The method of claim 33 further comprising energizing the ultrasonic probe to remove the biological material in the vasculature.

48. A method of moving an ultrasonic probe along a path in a vasculature of a body to ablate a biological material comprising:
engaging a flexible material having a high radiopacity to the ultrasonic probe;
inserting the ultrasonic probe with the flexible material into a vasculature;
advancing the ultrasonic probe within the vasculature; and
activating an ultrasonic energy source to provide an ultrasonic energy to the ultrasonic probe to ablate the biological material.

49. The method of claim 48 further comprising shaping the flexible material to increase a radial span of the ultrasonic probe within the vasculature.

50. The method of claim 48 wherein the flexible material cushions a tip of the ultrasonic probe to protect the vasculature when moving the ultrasonic probe through the vasculature.

51. The method of claim 48 wherein the flexible material protects the vasculature as the ultrasonic probe is moved through the vasculature.

52. The method of claim 54 wherein the flexible material reduces the stresses on the ultrasonic probe as the ultrasonic probe is navigated within the vasculature.

53. A method for adhering a flexible material to an ultrasonic medical device comprising:
providing the flexible material to be adhered to the ultrasonic medical device;
engaging the flexible material to the ultrasonic medical device;
heating the flexible material engaged to the ultrasonic medical device with a heat source causing the flexible material to melt; and
cooling the flexible material engaged to the ultrasonic medical device to adhere the flexible material to the ultrasonic medical device.

54. The method of claim 53 wherein the flexible material is a polymer.

55. The method of claim 53 wherein the flexible material comprises a high radiopacity.

56. The method of claim 53 further comprising pre-extruding the flexible material to a desired shape and size.

57. The method of claim 53 wherein the flexible material has a hollow channel through the flexible material.

58. The method of claim 53 wherein the flexible material is a solid material.

59. The method of claim 53 further comprising melting the flexible material before engaging the flexible material to the ultrasonic medical device.

60. The method of claim 53 wherein the ultrasonic medical device is an ultrasonic probe.

61. The method of claim 60 wherein the ultrasonic probe comprises titanium.

62. The method of claim 53 wherein the heat source is an oven.

63. The method of claim 53 wherein the heat source is a hot air system.

64. The method of claim 53 wherein the heat source is a heating block.

65. The method of claim 53 further comprising heat shrinking a polymer over the flexible material engaged to the ultrasonic medical device.

66. The method of claim 53 further comprising applying a heat shrink in an expanded state over the flexible material engaging the ultrasonic medical device prior to melting the flexible material.

67. The method of claim 53 further comprising placing the flexible material and the ultrasonic medical device in a mold.
